(19) United States
(12) Patent Application Publication
(10) Pub. No.: US 2016/0150943 A1
(43) Pub. Date: Jun. 2, 2016

(54) Digital Swappable Endoscope

(71) Applicant: CHIYI Technology, New Taipei City (TW)

(72) Inventor: Yen-Tsung Lin, New Taipei City (TW)

(21) Appl. No.: 14/953,267

(22) Filed: Nov. 27, 2015

(30) Foreign Application Priority Data
Nov. 27, 2014 (TW) 103141282

(51) Int. Cl. A61B 1/00; A61B 1/045; A61B 1/05

(52) U.S. Cl. CPC A61B 1/00006; A61B 1/051; A61B 1/00009; A61B 1/00045; A61B 1/00163; A61B 1/045

(57) ABSTRACT

A digital swappable endoscope including pipe end and host end is provided. The pipe end includes lens module, image sensor, and sensor processor. The lens module is configured to capture a light. The sensor processor is connected to the image sensor and transforms the light into a digital sign. The host end includes a programmable logic device and a CPU. The programmable logic device is connected to the pipe end and configured to receive the digital signal. The CPU is connected to the programmable logic device. The programmable logic device is configured to determine the signal format of the digital signal. The programmable logic device carries out the following step after determining:

A. if the signal format is conformed to specification, the digital signal is transmitted to the CPU; and

B. if the signal format is not conformed to specification, a simulation signal is transmitted to the CPU.

### BACKGROUND OF THE INVENTION

1. Field of the Invention

The invention relates to an endoscope, and more particularly relates to a digital swappable endoscope.

2. Description of the Prior Art

In the development of medical science, the endoscope is an important technology for medical research or clinical diagnosis. The physician can use the endoscope to enter patient's organ such as oesophagus, duodenum or large intestine, etc. Thus, the physician can observe the status inside patient's body via the lens of the endoscope. Moreover, the endoscope further can be used with small cutting tools, so the physician can use the small cutting tools to remove the lesion for treating some disease. Comparing with traditional surgery, the surgery using endoscope does not make huge pressure for patient on physiology or psychology.

Please refer to FIG. 1. The FIG. 1 shows the architecture chart of endoscope 100 in the prior art. The endoscope 100 includes a pipe end 110 and host end 120. The pipe end 110 is configured to capture the light 10 via a lens module, and the light 10 would be transformed into an image signal by an image sensor 120 and a sensor processor 113. The code used at transforming process is programmed in the MCU 114. In other words, the pipe end 110 is driven by the MCU 114. The image signal would be transmitted to host end 120 via a wire 115 and a connector 116, and the host end 120 receives the image signal via the connector 121. The image signal is operated by an image processor 122 and a CPU 123 and the image would be displayed at a display device 124 or saved in the storage device 125.

However, the above architecture of the endoscope 100 has some disadvantages. The user cannot adjust detailed option of the image sensor 112 from the host end 120 because most of the codes are programmed in the micro processor 114. In addition, if the image sensor 112 needs to output signal having different format, the micro-processor 114 and the motherboard of the pipe end 110 should be changed, and it is expensive. Beside, when the MCU failure or oscillator (not drawing in FIG. 1) error is happened because of short-circuit or electromagnetic interference, the host end 120 cannot be restarted, and the image sensor 112 also cannot sense the image (named as crash). Moreover, in the traditional architecture, if the pipe end 110 is removed during the period of transmitting image, the image packet transmitting would be interrupted. However, the host end 120 is still waiting to receive the image packet and stop working, and then the host end 120 would be crashed.

Therefore, how to design a new architecture of the endoscope which is stable, hard to crash and allowed to play a plurality of format of image. It is worth considering to a person having ordinary skills in the art.

### SUMMARY OF THE INVENTION

One object of the present invention is to provide a digital swappable endoscope which can transform signal format and stable, hard to crash. To achieve the foregoing and other objects, a digital swappable endoscope is provided. The digital swappable endoscope includes a pipe end and a host end. The pipe end includes a lens module, an image sensor, and a sensor processor. The lens module is configured to capture a light. The sensor processor is connected to the image sensor and transforms the light into a digital sign. The host end includes a programmable logic device and a CPU.

The programmable logic device is connected to the pipe end and configured to receive the digital signal. The CPU is connected to the programmable logic device. The programmable logic device is configured to determine identify the signal format of the digital signal. The programmable logic device carries out the following steps after determining:

A. if the signal format is conformed to at least one predetermined specification, the digital signal is transmitted to the CPU; and

B. if the signal format is not conformed to the predetermined specification, a simulation signal is transmitted to the CPU.

In the above digital swappable endoscope, wherein, the programmable logic device transmits a re-initializing signal to the photosensitive chip if the image format is not conformed to the predetermined specification.

In the above digital swappable endoscope, wherein, the programmable logic device transforms the signal format if the image format is conformed.

In the above digital swappable endoscope, wherein, the host end further comprises an image processor disposed between the CPU and the programmable logic device.

In the above digital swappable endoscope, wherein, the host end further comprises a display device connected to the CPU.

In the above digital swappable endoscope, wherein, when the CPU receives the simulation signal, a non-signal screen is displayed on the display device.

In the above digital swappable endoscope, wherein, the host end further comprises a storage device connected to the CPU.

In the above digital swappable endoscope, wherein, the programmable logic device is a complex programmable logic device.

In the above digital swappable endoscope, wherein, the programmable logic device is a field-programmable gate array.

In the above digital swappable endoscope, wherein, the image format conformed to the specification is MIPI, LVDS, Sub LVDS, RAW or UVC.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 shows the architecture chart of traditional endoscope.

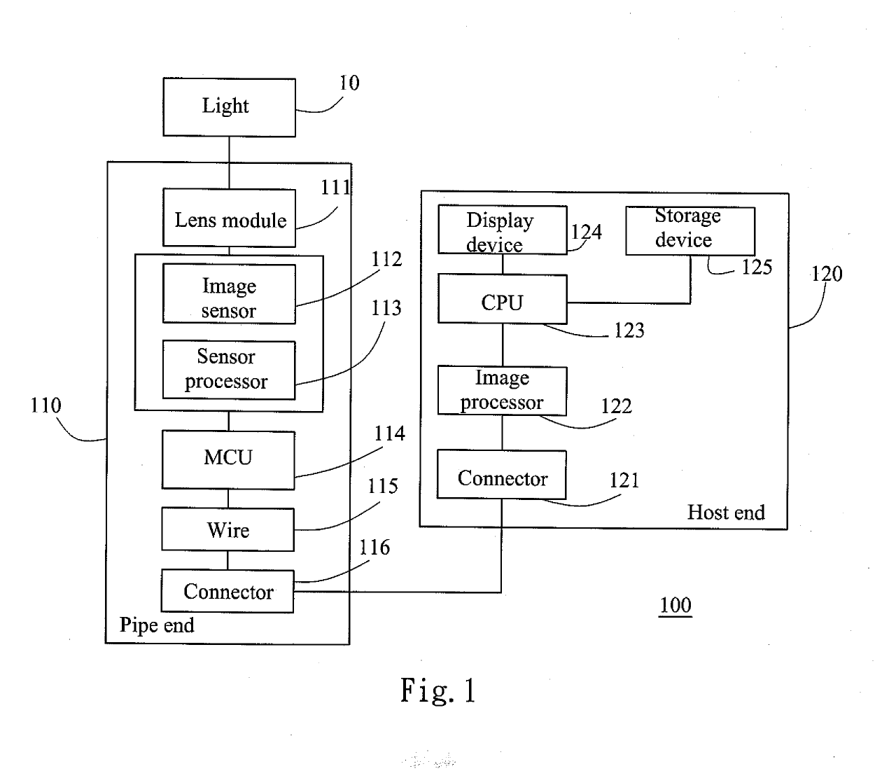

FIG. 2 shows the architecture chart of digital swappable endoscope.

### DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiment illustrated.

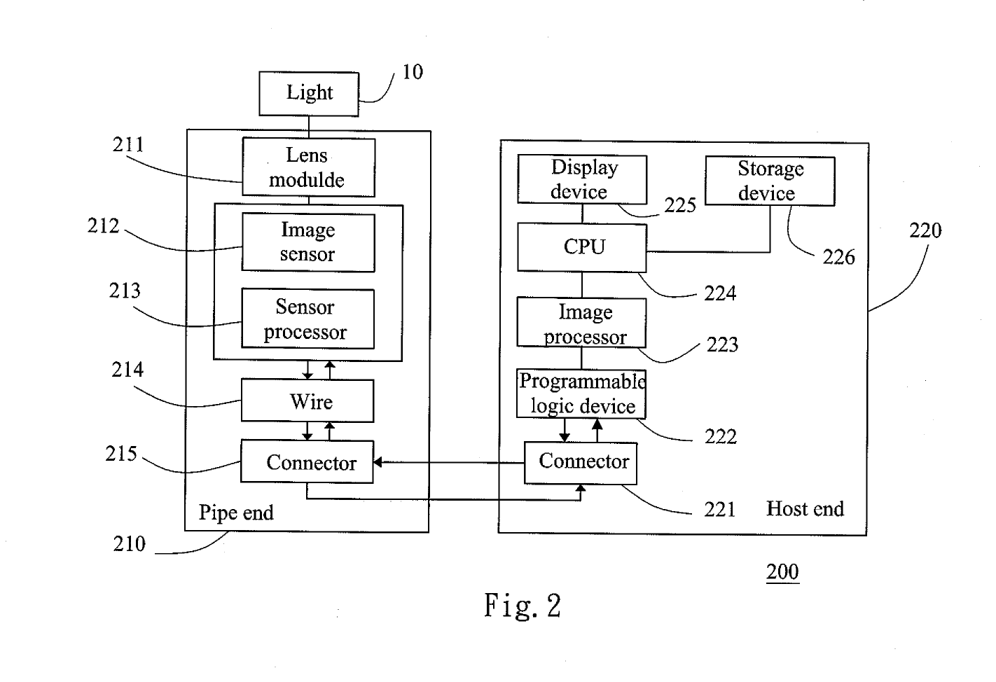

Please refer FIG. 2. FIG. 2 shows the architecture chart of digital swappable endoscope 200 of the present invention. Digital swappable endoscope 200 includes a pipe end 210 and a host end 210. A lens module 211 is disposed on one end of the pipe end 210 and configured to capture a light 10. A connector 215 is disposed on another end of the pipe end 210 and connected to the connector 221 of the host end 220. The lens module 211 is connected to a photosensitive chip which includes an image sensor 212 and a sensor processor 213. In this embodiment, the image sensor 212 is

Complementary Metal-Oxide Semiconductor (CMOS). The image sensor 212 is connected to the lens module 211 and configured to sense light 10 captured by the lens module 211. The image sensor 212 is further connected to the sensor processor 213. The sensor processor 213 is configured to transform the light 10 sensed by the image sensor 212 into a digital signal. Beside, by the sensor processor 213 the parameters of the image sensor 212 can be adjusted, and the parameters are such as initial, aperture, frame rate, and white balance, etc. After the light 10 is transformed into the digital signal, the digital signal would be transmitted to the host end 220 via a wire 214 and the connector 215. The wire 214 is a flexible wire which connects photosensitive chip and connector 215 and the connector 215 of the pipe end 210 is connected to the connector 221 of the host end 220. Thus, the image captured by the pipe end 210 can be transmitted to the host end 220 for subsequent processing.

The digital signal would be transmitted to a programmable logic device 222 after the digital signal is received by the connector 221 of the host end 220. The programmable logic device is such as a Complex Programmable Logic Device (CPLD) or Field-Programmable Gate Array (FPGA). The programmable logic device 222 is configured to determine the signal format of the digital signal and control the photosensitive chip. The detail of the programmable logic device 222 would be explained later. After passing through the programmable logic device 222, the digital signal would be transmitted to the image processor 223. The image processor 223 is such as Graphics Processing Unit (GPU) and connected between the CPU 224 and the programmable logic device 222. The transformed image would be transmitted to the display 225 for playing or to the storage device 226 for saving after processed by CPU 224.

The programmable logic device 222 is configured to determine whether the signal format is conformed to at least one predetermined specification or not and keep the digital swappable endoscope 200 working to avoid crash or working stoppage. In this embodiment, the image format conformed to the specification is such as MIPI, LVDS, Sub LVDS, RAW or UVC. Moreover, a bidirectional transmission between the programmable logic device 222 and the photosensitive chip can be achieved by Inter-Integrated Circuit (I2C). After the programmable logic device 222 identifies the signal format of the digital signal, the programmable logic device 222 would carry out the following step:

A: If the signal format is conformed to the specification, the programmable logic device 222 will transmit the digital signal to the CPU 224 from the image processor 223. At this moment, the digital signal would be processed by the CPU 224. Then, the processed digital signal can be displayed on the display device 225 or transmitted to and saved in the storage device 226 for searching and watching in the future. In addition, the programmable logic device 222 is further configured to transform the signal format of the digital signal. The signal format can be transformed into the other format conformed to specification based on the user's setting for unifying the signal format of the digital signal saved in the storage device 226.

B: if the signal format is not conformed to the specification, the programmable logic device 226 would produce and transmit a simulation signal to the CPU 224. The simulation signal can avoid the host end 220 producing error and crashed when the host end 220 receives the signal not conformed to the specification. The display device 225 would display a screen of “No signal” when the CPU 224 receives the simulation signal. Simultaneously, a re-initializing signal would be transmitted to the photosensitive chip by the programmable logic device 222 in order to restart the photosensitive chip and keep the image being transmitted.

Moreover, there are many behavior instructions saved in the programmable logic device 222 such as initial, aperture, frame rate, and white balance, etc. Therefore, the user can directly adjust the parameter of the sensor processor 213 on the host end 220 because of these behavior instructions.

Comparing with the architecture of traditional endoscope in the prior art, the digital swappable endoscope 200 of the present invention has following advantage:

1. The programmable logic device 222 is disposed on the host end 220. The user can adjust the parameter of the photosensitive chip on the host end 220 based on the user's demand or the screen of the display device 225.

2. The programmable logic device 222 can be designed for pipe end 210 having different photosensitive chip or signal format. Beside, the programmable logic device 222 also can be designed to support a plurality of signal format. The part of the host end 220 is not needed to be replaced when the pipe end 210 is replaced. It can obviously reduce the cost of the endoscope.

3. If the processor failure or oscillator error is happened because of the short-circuit or electromagnetic interference, the photosensitive chip would be restart by programmable logic device 222 to keep the image transmitting.

4. The signal from pipe end 210 would be filtered by programmable logic device 222. If the programmable logic device 222 receives the signal not conformed to specification or cannot receive the signal, the programmable logic device 222 still transmits signal to the CPU 224 to keep host end 220 working instead of crashing. Beside, although the pipe end 210 is removed, the programmable logic device 222 still transmits signal to the CPU 224. Thus, the host end 220 would not crash when the pipe end 210 is removed, and the pipe end 210 has the advantage of hot plugging.

Those skilled in the art will readily observe that numerous modifications and alternatives of the device and method may be made while retaining the teachings of the invention. Accordingly, the above disclosure should be construed as limited only by the meters and bounds of the appended claims.

I claim:

1. A digital swappable endoscope comprising:
a pipe end comprising:
a lens module configured to capture a light; and
a photosensitive chip comprising:
an image sensor connected to the lens module and sensing the light; and
a sensor processor connected to the image sensor and transforming the light into a digital signal; and
a host end connected to the pipe end, the host end comprising:
a programmable logic device connected to the photosensitive chip and receiving the digital signal; and
a CPU connected to the programmable logic device;
wherein the programmable logic device is configured to identify the signal format of the digital signal, the programmable logic device carrying out a plurality of steps after determining:
A. if the signal format is conformed to at least one predetermined specification, transmitting the digital signal to the CPU; and
B. if the signal format is not conformed to the predetermined specification, transmitting a simulation signal to the CPU.

2. The digital swappable endoscope of claim 1, wherein the programmable logic device transmits a re-initializing signal to the photosensitive chip if the image format is not conformed to the predetermined specification.

3. The digital swappable endoscope of claim 1, wherein the programmable logic device transforms the signal format if the image format conformed.

4. The digital swappable endoscope of claim 1, wherein the host end further comprises an image processor disposed between the CPU and the programmable logic device.

5. The digital swappable endoscope of claim 1, wherein the host end further comprises a display device connected to the CPU.

6. The digital swappable endoscope of claim 5, wherein when the CPU receives the simulation signal, a non-signal screen is displayed on the display device.

7. The digital swappable endoscope of claim 1, wherein the host end further comprises a storage device connected to the CPU.

8. The digital swappable endoscope of claim 1, wherein the programmable logic device is a complex programmable logic device.

9. The digital swappable endoscope of claim 1, wherein the programmable logic device is field-programmable gate array.

10. The digital swappable endoscope of claim 1, wherein the image format conformed to the specification is MIPI, LVDS, Sub LVDS, RAW or UVC.
